(19) United States
(12) Patent Application Publication
(10) Pub. No.: US 2024/0102081 A1
(43) Pub. Date: Mar. 28, 2024

(54) IDENTIFYING DISEASE-CAUSING HUMAN DDX41 GENETIC VARIANTS

(71) Applicant: Wisconsin Alumni Research Foundation, Madison, WI (US)

(72) Inventors: Emery Bresnick, Madison, WI (US); Jeong-Ah Kim, Madison, WI (US)

(21) Appl. No.: 18/471,461

(22) Filed: Sep. 21, 2023

Related U.S. Application Data
(60) Provisional application No. 63/409,311, filed on Sep. 23, 2022.

(51) Int. Cl. C12Q 1/6809; C12N 5/071; C12Q 1/6883

(52) U.S. Cl. CPC C12Q 1/6809; C12N 5/0672; C12Q 1/6883; C12N 2503/00; C12N 2510/04; C12Q 2600/118

(57) ABSTRACT

Described herein is an in vitro genetic rescue assay for identifying a functionally defective DDX41 variant which includes identifying a DEAD-Box Helicase 41 (DDX41) variant of uncertain significance (VUS), infecting a first Ddx41^{+/-} cell with a retrovirus expressing the DDX41-VUS, infecting a second Ddx41^{+/-} cell with a retrovirus expressing a wild type control DDX41, growing the first and second infected cells in culture for a period of time and quantitating mRNA expression of a DDX41-regulated transcript in both the first and second infected cells after the period of time, calculating a differential expression of the DDX41-regulated transcript for the first infected cell compared the second infected cell, and identifying the DDX41-VUS as the functionally defective DDX41 variant wherein a change in the differential expression is 1.5-fold or greater. The identified functionally defective DDX41 variants can be used in methods of monitoring a patient for the development/progression of myeloid malignancy.

### CROSS-REFERENCE TO RELATED APPLICATIONS

This application claims priority to U.S. Provisional Application 63/409,311 filed on Sep. 23, 2022, which is incorporated herein by reference in its entirety.

SEQUENCE LISTING

The Instant Application contains a Sequence Listing which has been submitted electronically in XML format and is hereby incorporated by reference in its entirety. Said XML copy, created on Sep. 18, 2023 is named SEQ_LIST-107668_004 and is 49.9 KB (51,124 bytes) in size. The Sequence Listing does not go beyond the disclosure in the application as filed.

### FIELD OF THE DISCLOSURE

The present disclosure is related to an in vitro genetic rescue assay for identifying functionally defective DDX41 variants, and the use of the identified variants to monitor a patient for the development/progression of a myeloid malignancy.

### BACKGROUND

Myeloid malignancies are malignant clonal hematopoietic stem cell disorders characterized by ineffective hematopoiesis, marrow dysplasia, and/or peripheral blood cytopenia. Myelodysplastic Syndromes (MDS) are disorders characterized by a failure to produce sufficient healthy, mature blood cells, which have a high propensity for transformation into acute myeloid leukemia (AML). Acute myeloid leukemia (AML) originates from the transformation and clonal expansion of undifferentiated hematopoietic progenitors, characterized by altered growth, differentiation, and proliferation capacities, which result in failure of bone marrow hematopoietic functions. Patients with AML frequently experience infections, easy bleeding, anemia, and organ failure for example.

DEAD-box helicase 41 (DDX41) is a highly conserved member of the RNA helicase family of metazoan proteins that exerts post-transcriptional and translational functions, including the regulation of pre-mRNA splicing and ribosomal RNA transcription. DDX41 senses intracellular DNA in the cGAS-STING pathway, maintains genomic stability at transcriptional loci harboring R-loops regulates inflammatory gene expression and controls snoRNA processing in mouse leukemic stem/progenitor cells.

Heterozygous DDX41 germline variants generate a predisposition for the development of MDS and acute myeloid leukemia AML. Patients present with cytopenias, bone marrow hypocellularity, erythroid dysplasia and/or myeloid malignancy. Although DEAD-box helicase 41 (DDX41) is implicated in oncogenic and innate immune mechanisms, there are many unanswered questions about how the ever-increasing spectrum of genetic variants impact DDX41 activity. Assays to discriminate DDX41 variants of undetermined significance from pathogenic variants, which is fundamental for clinical genetic curation, have not been reported, thus limiting the clinical utility of genetic information.

What is needed are methods of identifying DDX41 pathogenic germline variants.

### BRIEF SUMMARY

In an aspect, an in vitro genetic rescue assay for identifying functionally defective DDX41 variants comprises
identifying a DEAD-Box Helicase 41 (DDX41) variant of uncertain significance (VUS),
infecting a first Ddx41^{+/-} cell with a retrovirus expressing the DDX41-VUS,
infecting a second Ddx41^{+/-} cell with a retrovirus expressing a wild type control DDX41,
growing the first and second infected cells in culture for a period of time and quantitating mRNA expression of a DDX41-regulated transcript in both the first and second infected cells after the period of time, wherein the DDX41-related transcript comprises Sdc1, Fam133b, Gas5, Clk3, or a combination thereof,
calculating a differential expression of the DDX41-regulated transcript for the first infected cell compared to the second infected cell, and
identifying the DDX41-VUS as the functionally defective DDX41 variant wherein a change in the differential expression is 1.5-fold or greater,
wherein a Ddx41^{+/-} cell has a DDX41 expression level that is reduced by at least 50% compared to that of a Ddx41^{+/+} cell.

In another aspect, a method of monitoring a patient for the development/progression of a myeloid malignancy comprises
determining the presence or absence of a functionally defective DDX41 variant determined according to the method described above in a sample from the patient,
optionally determining the presence or absence of the functionally defective DDX41 variant in one or more family members of the patient, and
monitoring the patient and optionally the one or more family members for the development of one or more symptoms of myeloid malignancy when the patient and the one or more family members carry the functionally defective DDX41 variant.

In a further aspect, a method of identifying a patient as at risk for the development/progression of a myeloid malignancy comprises identifying a functionally defective DDX41 variant in the germline of the patient, wherein the functionally defective DDX41 variant is Lys331del.

### BRIEF DESCRIPTION OF THE DRAWINGS

FIGS. 1A-E show family histories and structural predictions of myeloid malignancy associated DDX41 variants. FIG. 1A shows K33 ldel and R293H pedigrees. Probands are indicated with an arrow. FIG. 1B shows the domain structure of human DDX41. Germline and somatic mutations are indicated within the corresponding domains. FIG. 1C shows Sanger sequencing data of probands in each family FIG. 1D provides the predicted structure of mutant human DDX41 by AlphaFold and 3-D images. FIG. 1E shows the confidence of predicted structure from the AlphaFold analysis.

FIGS. 2A-J show the quantitative genetic rescue assay discriminates DDX41 and human myeloid malignancy-associated variant activities. FIG. 2A illustrates the genetic rescue system. Three CRISPR/Cas9 RNP complexes for Ddx41 were electroporated individually into HoxB-immortalized (hi)-progenitors, and clonal lines were isolated. Retrovirus expressing GFP or GFP and DDX41 or a human disease-associated DDX41 variant were infected into hi-Ddx41^{+/+} and hi-Ddx41^{+/-} cells. GFP-positive cells were isolated by flow cytometry and analyzed by qRT-PCR and RNA-seq. Protein was analyzed by semi-quantitative western blotting. FIG. 2B illustrates exons corresponding to representative domains and target loci of crRNAs on murine Ddx41. FIG. 2C shows Sanger sequencing of genomic DNA at the edited region of hi-Ddx41^{+/-} clonal lines. The crRNA used to generate each clone is indicated in parenthesis. FIG. 2D shows DDX41 protein levels in hi-Ddx41^{+/+} and hi-Ddx41^{+/-} cells were analyzed by semi-quantitative Western blotting using two different antibodies (mouse monoclonal anti-DDX41 antibody (2F4), Novus Biologicals, and a rabbit polyclonal anti-DDX41 antibody). Each clone number indicated corresponds to each clone in

FIG. 2C. FIG. 2E shows representative Western blots of Flag-tagged DDX41 and variants and endogenous DDX41 using anti-DDX41 antibody in genetic rescue assay. FIG. 2F shows quantitative analysis of DDX41 protein of FIG. 2E (n=3 per group). FIG. 2G shows the subcellular localization of DDX4 land variants by confocal fluorescence microscope using anti-Flag antibody. FIG. 2H shows mRNAs regulated by DDX41 genetic rescue in hi-Ddx41^{+/-} cells. Heatmap with TPM values of DDX41-regulated mRNAs from total RNA-Seq (n=3 per group). FIG. 21 shows qRT-PCR analysis of DDX41-regulated mRNAs (n=6 per group). Statistics were calculated using one-way ANOVA, followed by Tukey's test; *, P≤0.05; **, P≤0.01; ***, P≤0.001; ****, P≤0.0001. FIG. 2J shows monocytic and granulocytic populations of live GFP+ cells with CD11b and CD115 surface markers detected by flow cytometry (n=6 per group). Statistics were calculated using one-way ANOVA, followed by Tukey's test; *, P<0.05; **, P<0.01; ***, P<0.001; ****, P<0.0001.

FIGS. 3A-D show qRT-PCR analysis of DDX41-regulated primary transcripts of Clk3 (n=4 per group). qPCR primers on adjacent exons and introns were indicated with arrows in FIG. 3A. Transcript levels were measured in myeloid progenitors (hi-Ddx41^{+/-}; FIGS. 3B,D) and pro-erythroblasts (G1E-ER-G1-Ddx41^{+/-}; FIG. 3C). Statistics were calculated using one-way ANOVA, followed by Tukey's or Sidak test;*, P≤0.05;**, P≤0.01; ***, P≤0.001; ****, P≤0.0001.

The above-described and other features will be appreciated and understood by those skilled in the art from the following detailed description, drawings, and appended claims.

### DETAILED DESCRIPTION

Described herein a genetic rescue assay that provided the identification of two rare DDX41 variants of uncertain significance in two unrelated families as myeloid malignancy-associated DDX41 genetic variants. The genetic rescue assay quantitatively discriminates activities of DDX41 and myeloid malignancy-associated DDX41 genetic variants. The analyses described herein revealed that the variants were impaired in their intrinsic RNA-regulatory activities and to induce monocytic differentiation markers. The quantitative assay described herein can be leveraged to elucidate mechanisms and interventions that promote and/or oppose DDX41 function, thereby influencing DDX41-linked pathogenicity.

In an aspect, an in vitro genetic rescue assay for identifying functionally defective DDX41 variants comprises
identifying a DEAD-Box Helicase 41 (DDX41) variant of uncertain significance (VUS),
infecting a first Ddx41^{+/-} cell with a retrovirus expressing the DDX41-VUS,
infecting a second Ddx41^{+/-} cell with a retrovirus expressing a wild type control DDX41,
growing the first and second infected cells in culture for a period of time and quantitating mRNA expression of a DDX41-regulated transcript in both the first and second infected cells after the period of time, wherein the DDX-41 related transcript comprises Sdc1, Fam133b, Gas5, Clk3, or a combination thereof,
calculating a differential expression of the DDX41-regulated transcript for the first infected cell compared the second infected cell, and
identifying the DDX-41-VUS as the functionally defective DDX41 variant wherein a change in the differential expression is 1.5-fold or greater.

In an aspect, a wherein a Ddx41^{+/-} cell has a DDX41 expression level that is reduced by at least 50% compared to that of a Ddx41^{+/+} cell.

As used herein, a VUS is a variant or unknown or uncertain significance, that is, a genetic variant identified through genetic testing for which the association with disease risk/progression is unclear. A VUS can be benign or pathogenic, for example, and may be identified in a large or small number of individuals. In an aspect, the VUS can be identified in a patient presenting with cytopenia, bone marrow hypocellularity, erythroid dysplasia, and/or myeloid malignancy, but not diagnosed with acute myeloid leukemia.

Cytopenia, for example, is a condition in which one or more blood cell types are lower than normal. Patients who exhibit cytopenia are at risk of progressing to myeloid disease.

Bone marrow hypocellularity is decreased production in one or more hematopoietic cell lineages (myeloid or erythroid). Acute leukemias usually present with hypercellular bone marrow.

Erythroid dysplasia is a condition in which the erythroid cells in the bone marrow are abnormal in size, shape and/or number.

Myeloid malignancies are clonal diseases of hematopoietic stem or progenitor cells. Myeloid malignancies can be present in the bone marrow and in the peripheral blood.

“Acute myeloid leukemia” or “AML”, also known as “acute myelogenous leukemia”, has its general meaning in the art and refers to a cancer of the myeloid line of blood cells, characterized by the rapid growth of abnormal white blood cells that accumulate in the bone marrow and interfere with the production of normal blood cells. AML may be classified using either the World Health Organization classification or the FAB classification.

In an aspect, the DDX41 VUS is identified in a database such as ClinVar or gnomAD.

As described in the Examples, the inventors engineered Hoxb8-immortalized mouse fetal liver progenitor cells, which exhibit a normal myeloid progenitor cell phenotype with CRISPR-Cas9 to generate Ddx41^{+/-} clonal lines with reduced endogenous DDX41 protein levels. WT or mutant DDX41 proteins can be expressed in the Ddx41^{+/-} clonal lines at near-physiological levels and functional consequences e.g., impact on transcript levels. In an aspect, a Ddx41^{+/-} cell has a DDX41 expression level that is reduced by at least 50% compared to that of a Ddx41^{+/+} cell.

In the assay, a first Ddx41^{+/-} cell is infected with a retrovirus expressing the DDX41-VUS, and a second Ddx41^{+/-} cell is infected with a retrovirus expressing a wild type control DDX41. Infection can include standard techniques for infection of cells with a retrovirus. The cells are then grown in culture for a short period of time, such as 2 to 3 days, and then mRNA expression of a DDX41-regulated transcript in both the first and second infected cells after the period of time is quantitated. Based on the RNA-Seq experiments described herein, the DDX41-related transcript comprises Sdc1, Fam133b, Gas5, Clk3, or a combination thereof. A differential expression of the DDX41-regulated transcript for the first infected cell (transcript level in the presence versus absence of DDX41-VUS) is compared to that of the second infected cell (transcript level in the presence versus absence of wild type DDX41) and subjected to statistical analysis. The DDX41-VUS is identified as a functionally defective DDX41 variant wherein a change in the differential expression is 1.5-fold or less than, more specifically 1.8-fold, 2-fold or 2.5-fold less than the differential expression of achieved by expression of wild type DDX41.

In an aspect, the method further includes infecting a third Ddx41^{+/-} cell with a retrovirus expressing a functionally defective variant (e.g., G173R) control DDX41, growing the third infected cells in culture for a short period of time and quantitating mRNA expression of DDX41-regulated genes in the third infected cells, wherein the DDX41-regulated genes comprises Sdc1, Fam133b, Gas5, Clk3, or a combination thereof, and comparing the differential expression of the DDX41-regulated genes for the first infected cell to that of the third infected cell to establish values expected for a functionally defective control. The functionally defective control can be used to supplement the wild type control.

In an aspect, quantitating mRNA expression of a DDX41-regulated transcript comprises reverse-transcriptase PCR. Alternative techniques for quantitating mRNA expression include ribonuclease protection assays and RNA-sequencing.

In order to further verify the functionally defective DDX41 variants flow cytometry may be used. In an aspect, the method further comprises performing flow cytometry on the first infected cells and second infected cells and quantitating a monocytic marker of differentiation of hematopoietic progenitor cells and a granulocytic marker of differentiation of hematopoietic progenitor cells with marker-specific antibodies, wherein a 50% or greater increase in the quantitative level of monocytic marker and 30% or greater decrease in the quantitative level of the granulocytic marker in the first infected cells compared to the second infected cells identifies the functionally defective DDX41 variant as a pathogenic DDX41 variant.

In an aspect, the monocytic markers are CD11b⁺CD115⁺ and/or Ly6C⁺, and the granulocytic markers are CD11b⁺CD115⁻ and/or Ly6G⁺.

Advantageously, the functionally defective DDX41 variants identified by the methods described herein can be used to monitor patients for the development/progression of myeloid malignancy. An exemplary method includes determining the presence or absence of a functionally defective DDX41 variant determined according to the method described herein, optionally determining the presence or absence of the functionally defective DDX41 variant in one or more family members of the patient, and monitoring the patient and optionally the one or more family members for the development of one or more symptoms of myeloid malignancy when the patient and the one or more family members carry the functionally defective DDX41 variant. Exemplary myeloid malignancies include myelodysplastic syndromes (MDS) and acute myeloid leukemia (AML).

Exemplary individuals include those who have a risk factor for developing AML such as being older than 65, being a smoker, having an inherited genetic disorder such as Down syndrome and ataxia telangiectasia, having previously received radiation or chemotherapy, exposure to certain chemicals and having other blood/bone marrow disorders such as aplastic anemia and myelodysplastic syndromes. In addition, having a family member carrying a functionally defective DDX41 variant or other defective variant associated with AML or a family history of myeloid malignancy is also a risk factor.

Exemplary patient samples include blood, bone marrow, fibroblasts, or fractional bone marrow hematopoietic stem/progenitor cells.

Exemplary symptoms of myeloid malignancy include fever, fatigue, irregular heartbeat, dizziness, bone pain, frequent nosebleeds, bleeding and swollen gums, bruising on skin, loss of appetite, excessive sweating, shortness of breath, unexplained weight loss, headaches, diarrhea, menorrhagia, slurred speech, confusion, abdominal swelling, pale skin, seizures, vomiting, loss of balance, facial numbness, blurred vision, or a combination thereof.

In an aspect, wherein when the patient carries the functionally defective DDX41 variant, the method further comprises administering a treatment for the myeloid malignancy.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “chemotherapeutic agent” refers to any chemical agent with therapeutic usefulness in the treatment of cancer. Chemotherapeutic agents as used herein encompass both chemical and biological agents. These agents function to inhibit a cellular activity upon which the leukemic cell depends for continued survival. Categories of chemotherapeutic agents include alkylating/alkaloid agents, antimetabolites, hormones or hormone analogues, and miscellaneous antineoplastic drugs. Most if not all of these drugs are directly toxic to leukemic cells and do not require immune stimulation.

In some embodiments the chemotherapeutic agent is cytarabine (cytosine arabinoside, Ara-C, Cytosar-U®), quizartinib (AC220), sorafenib (BAY 43-9006), lestaurtinib (CEP-701), midostaurin (PKC412), carboplatin, carmustine, chlorambucil, dacarbazine, ifosfamide, lomustine, mechlorethamine, procarbazine, pentostatin, (2′deoxycoformycin), etoposide, teniposide, topotecan, vinblastine, vincristine, paclitaxel, dexamethasone, methylprednisolone, prednisone, all-trans retinoic acid, arsenic trioxide, interferon-alpha, rituximab (Rituxan®), gemtuzumab ozogamicin, imatinib mesylate, melphalan, busulfan (Myleran®), thiotepa, bleomycin, platinum (cisplatin), cyclophosphamide, (Cytoxan®), daunorubicin, doxorubicin, idarubicin, mitoxantrone, 5-azacytidine, cladribine, fludarabine, hydroxyurea, 6-mercaptopurine, methotrexate, 6-thioguanine, or any combination thereof.

In some embodiments, the chemotherapeutic agent is a Bcl-2 inhibitor. In some embodiments, the Bcl-2 inhibitor comprises 4-(4-{[2-(4-chlorophenyl)-4,4-dimethylcyclohex-1-en-l-yl]methyl}piperazin--1-yl)-N-({3-nitro-4-[(tetrahydro-2H-pyran-4-ylmethyl)amino]phenyl}sulfony-1)-2-(1H-pyrrolo [2,3-b]pyridin-5-yloxy)benzamide (also known as, and optionally referred to herein as, venetoclax, or ABT-199, or GDC-0199) or a pharmaceutically acceptable salt thereof.

In some embodiments, the chemotherapeutic agent is a FLT3 inhibitor. Examples of FLT3 inhibitors include N-(2-diethylaminoethyl)-5-[(Z)-(5-fluoro-2-oxo-1H-indol-3-ylidene)methyl]-2,4-dimethyl-1H-pyrrole-3-carboxamide, sunitinib, also known as SU11248, and marketed as SUTENT® (sunitinib malate); 4-[4-[[4-chloro-3-(trifluoromethyl)phenyl]carbamoylamino]phenoxy]-N-methy- 1-pyridine-2-carboxamide, sorafenib, also known as BAY 43-9006, marketed as NEXAVAR® (sorafenib); (9S,10R,11R,13R)-2,3,10,11,12,13-hexahydro-10-methoxy-9-methyl-11-(methyl- amino)-9,13-epoxy-1H,9H-diindolo [1,2,3-gh:3′,2′,1′-1m]pyrrolo[3,4-j][1,7]benzodiamzonine-1-one, also known as midostaurin or PKC412; (5 S,6 S,8R)-6-hydroxy-6-(hydroxymethyl)-5-methyl-7,8,14,15-tetrahydro-5H-16-oxa- -4b,8a,14-triaza-5,8-methanodibenzo[b,h]cycloocta[jkl]cyclopenta[e]-as-indacen-13 (6H)-one, also known as lestaurtinib or CEP-701; 1-(5-(tert-Butyl)isoxazol-3-yl)-3-(4-(7-(2-morpholinoethoxy)benzo[d]imida-zo[2,1-b]thiazol-2-yl)phenyl)urea, also known as Quizartinib or AC220; 1-(2-{5-[(3-methyloxetan-3-yl)methoxy]-1H-benzimidazol-1-yl}quinolin-8-yl-)piperidin-4-amine, also known as Crenolanib or CP-868,596-26

In some embodiments, the chemotherapeutic agent is an IDH (isocitrate dehydrogenase) inhibitor. In some embodiments, the IDH inhibitor is a member of the oxazolidinone (3-pyrimidinyl-4-yl-oxazolidin-2-one) family, and is a specific inhibitor of the neomorphic activity of IDH1 mutants and has the chemical name (S)-4-isopropyl-3-(2-(((S)-1-(4 phenoxyphenyl)ethyl)amino)pyrimidin-4-yl)oxazolidin-2-one.

Another treatment for AML is a stem cell transplant. In certain aspects, stem cell transplants allow for higher doses of chemotherapeutic agents to be administered. Exemplary stem cell transplants include allogenic stem cell transplants (typically from a tissue matched donor) and autologous stem cell transplants (a patient's own stem cells).

In a specific aspect, a method of identifying a patient as at risk for the development/progression of a myeloid malignancy comprises identifying a functionally DDX41 variant in the germline of the patient, wherein the functionally defective DDX41 variant is Lys331del.

In an aspect, the method further comprises administering a treatment for the myeloid malignancy such as chemotherapy or a stem cell transplant.

The invention is further illustrated by the following non-limiting examples.

### EXAMPLES

### Methods

Patients and data collection: Families 1 and 2 were identified in University of Wisconsin/UW Health Hereditary Hematology and Bone Marrow Failure Clinic. Informed consent was obtained from probands and available close relatives or their healthcare proxy for deceased individuals. DDX41 germline variants were identified through CLIA-certified clinical genetic testing and confirmed and segregated in available family members via Sanger sequencing. Medical and family history were obtained by patient and family member interviews and medical record review. This study was approved by University of Wisconsin-Madison Health Sciences Institutional Review Board and conducted in accordance with the Declaration of Helsinki.

Protein structure prediction: AlphaFold was used to predict DDX41 structure. The DDX41 model was obtained from AlphaFold database (AF-Q9UJV9). DDX41 mutant structural models were generated with AlphaFold v2.1.0 Colab.

HoxB8-immortalized (hi)-Ddx41 heterozygous mutant (Ddx41+/−) cells: To generate HoxB8-immortalized (hi)-Ddx41 mutant progenitors, CRISPR/Cas9 RNP complexes were introduced individually by electrophoresis into hi-WT progenitors 318 described in the art. ER-HoxB8-immortalized (hi)- hematopoietic progenitors were generated by retroviral infection of estrogen-regulated HoxB8 into primary Lin- cells isolated from E14.5 mouse fetal liver. The sex of the cells was male, identified by PCR analysis of the DDX3y gene with genomic DNA. For targeting Ddx41, three crRNAs (A;5′-AGATGAGGACGACATCCCGC-3′ (SEQ ID NO: 1), B;5′-TGATCGGCATTGCCTTCACG-3′ (SEQ ID NO: 2), and C;3′-ATGCTCAGGACATAACGCGG-5′ (SEQ ID NO: 3), Integrated DNA Technologies; Coralville, IA, USA) targeting distinct exons were annealed to tracrRNA to generated guide RNAs which were then assembled into RNP complexes with sNLS-spCas9-sNLS (Aldevron (Madison, WI, USA). Each RNP complex was introduced into 2×10⁵ hi-progenitor cells using 4D Nucleofector™ with P3 Primary Cell 4D-Nucleofector® X Kit (Lonza; Basel, Switzerland). 72 hours post-electroporation, clones were isolated by limiting dilution (0.5 cell/well of a 96-well plate). Clones were cultured until colonies became visible and where then transferred to larger wells. Sanger sequencing of genomic DNA was used to establish genotype, and sequencing data was analyzed by SnapGene Viewer (San Diego, CA, USA). Cells were cultured in OPTI-MEM™ supplemented with 10% FBS, 1% penicillin streptomycin, 1% SCF-conditioned medium, 28.6 μM β-mercaptoethanol, 1 μM estradiol, and 400 μg/ml G418 in a humidified 5% CO₂ incubator at 37° C.

DDX41 rescue assay: DDX41 and human myeloid malignancy-associated DDX41 variants were expressed from the MSCV-PIG retroviral expression vector. Retroviruses were packaged in 293T cells and retrovirus-containing supernatants were collected 48h post-transfection. hi-Ddx41^{+/+} and hi-Ddx41^{+/-} hematopoietic progenitors were infected with retrovirus expressing GFP alone or GFP and DDX41 (WT or variants). Cells were transferred to IMDM containing 2%1-BS and incubated with infectious supernatant by spinoculation for 90 min at 2,800 rpm at 30° C. Cells were cultured for 2 days in immortalized cell culture media described above. GFP+ cells were isolated by fluorescence-activated cell sorting with FACSAria™ cell sorter (BD Biosciences; Franklin Lake, NJ, USA). RNA and protein of sorted GFP-positive cells was analyzed by RNA-seq, RT-qPCR and semi-quantitative Western blotting.

Semi-quantitative Western blotting: Cells were washed with ice-cold PBS and boiled for 10 mM in 2× sodium dodecyl sulfate (SDS) lysis buffer (50 mM Tris (pH 6.8), 2% β-mercaptoethanol, 2% SDS, 0.1% bromophenol blue, 10% glycerol). Proteins were analyzed by western blotting with ECL 2 (Thermo Fisher Scientific; Waltham, MA, USA) and West Femto (Thermo Fisher Scientific) with rabbit monoclonal anti-Flag (Cell Signaling Technology; Danvers, MA, USA), mouse monoclonal anti-DDX41 (Novus Bioscience; Littleton, CO, USA), rabbit polyclonal anti-DDX41 antibody generated against purified full-length DDX41 protein (Antigen designed by inventors, produced by Cocalico Biologicals, Inc.; Stevens, PA, USA), mouse monoclonal anti-beta-actin (Cell Signaling Technology) or anti-GAPDH (Cell Signaling Technology). Blots were developed using LI-COR Odyssey® Imaging System (LI-COR Biosciences; Lincoln, NE, USA) and quantified by Image Studio Lite (version 5.2.5) (LI-COR Biosciences). Quantification is represented as mean with SD. Statistical analyses were conducted using ANOVA tests (significance cutoff of p value <0.05) as calculated using Prism software (GraphPad Software; San Diego, CA, USA).

Immunofluorescence: hi-Ddx41^{+/+} cells were infected with empty retrovirus or retroviruses expressing DDX41 or variants, collected on poly-L-lysine coated slides (Electron Microscopy Sciences; Hatfield, PA, USA) and fixed with 3.7% paraformaldehyde in PBS for 10 min at room temperature. Slides were washed with PBS and permeabilized with 0.2% Triton™ X-100 for 10 mM at room temperature. Washed slides were blocked with 3% BSA in PBS containing 0.1% Tween™ 20 for 1 h at room temperature and incubated with rabbit anti-Flag (Cell Signaling Technology) in 3% BSA at 4° C. overnight. After washing, slides were incubated with Alexa Fluor® 594 secondary antibody (Invitrogen; Waltham, MA, USA) for 1 h at room temperature, washed and mounted using Vectashield® mounting medium with DAPI (Vector Laboratories; Burlingame, CA, USA). Images were acquired with a Nikon A1R-S confocal microscope (Nikon; Minato City, Tokyo, Japan).

RNA-seq with DDX41 rescue assay: Three biological replicates of hi-Ddx41^{+/+} cells infected with empty vector and hi-Ddx41^{+/-} cells infected with empty vector or retroviruses expressing DDX41 or G173R or R525H were harvested and sorted for GFP+ cells on a FACSAria™ II instrument (BD Biosciences). RNA was purified using an RNAeasy® Micro Kit (Qiagen; Hilden, Germany) Library sequencing through Illumina® TruSeq™ Stranded Total RNA (rRNA reduction) was conducted by the University of Wisconsin-Madison Gene Expression Center and sequenced using an Illumina® NovaSeg™ 6000. Reads were aligned by STAR (version 2.5.2b) to the mouse genome (version mm39) with GENCODE basic gene annotations (version M22). Gene expression levels were quantified by RSEM (version 1,3.0), and differential expression was analyzed by edgeR (version 3.30,3).

qRT-PCR: Total RNA was purified from 2-5×10⁵ cells with TRIzol™ (Invitrogen) and 1-2 μg RNA was treated with DNase I (Thermo Fisher Scientific) for 15 min at room temperature. After heat inactivation of DNase I with EDTA for 10 min at 65° C., 0.5-1 μg RNA was incubated with a 4:1 mixture of oligo(dT) primers and random hexamer at 68° C. for 10 min. RNA/primers were incubated with Moloney murine leukemia virus reverse transcription (M-MLV RT) (Thermo Fisher Scientific), 5× first strand buffer (Thermo Fisher Scientific), 10 mM dithiothreitol (Thermo Fisher Scientific), RNAsin® (Promega), and 0.5 mM deoxynucleoside triphosphates (New England Biolabs; Ipswich, MA, USA) at 42° C. for 1 h and then heat inactivated at 95° C. for 5 mM. Quantitative gene expression analyses were conducted by real-time RT-PCR using Power SYBR™ Green Master Mix (Applied Biosystems; Waltham, MA, USA) and analyzed on a ViiA™ 7 Real-Time PCR System (Applied Biosystems). Control reactions without M-MLV RT yielded little to no signal. Relative expression of mRNA was determined from a standard curve of serial dilutions of cDNA samples and values were normalized to Hprt RNA expression. Quantitative RT PCR results were presented as box and whisker plots. Statistical comparisons were conducted using ANOVA test (significance cutoff of P value <0.05) as calculated using Prism software (GraphPad Software).

Cell differentiation and flow cytometry: hi-Ddx41^{+/+} and hi-Ddx41^{+/-} cells were infected with retroviruses expressing DDX41 or variants. One day post-infection, cells were washed with ice-cold PBS and resuspended in differentiation medium (OPTI-MEM™ supplemented with 10% FBS, 1% penicillin streptomycin, 1% SCF-conditioned medium, 1% IL-3-conditioned medium, and 28.6 pM beta-mercaptoethanol). Cells were cultured for 3 days at 37° C. For analysis of monocytic and granulocytic populations by flow cytometry, cells were washed with ice-cold PBS containing 2% FBS and 2 mM EDTA, and live/dead cell staining was conducted using Ghost Dye™ Red 780 (Tonbo Biosciences, San Diego, CA, USA) at 4° C. for 15 mM, followed by washing with PBS containing 2% FBS and 2 mM EDTA. Surface antigens were stained using 1:200 diluted combinations of APC-CD11b (BioLegend) and PE-CD115 (BioLegend) in PBS with 2% PBS and 2 mM EDTA at 4° C. for 30 min. After staining, cells were washed with ice-cold PBS containing 2% FBS and EDTA and analyzed on an Attune™ NxT Flow Cytometer (Thermo Fisher Scientific). Differentiated cell populations were analyzed using FlowJo v10.8.0 software (BD Biosciences).

TABLE 1
PRIMERS

| Target | Sequence | SEQ ID NO: |
|---|---|---|
| DDX41 Exon9-Forward | ATCATCTGCCCCTCGGTAAG | 4 |
| DDX41 Exon9-Reverse | CCCCAACTAACCTCCCCATT | 5 |
| DDX41 Exon10-Forward | ATACATAGGGCAGGTGGTGG | 6 |
| DDX41 Exon10-Reverse | AGCTGAGCAACTGAGACACA | 7 |
| Sdc1-exon3_4-Forward | GAGGATGGAACTGCCAATCA | 8 |
| Sdc1-exon3_4-Reverse | CCCAGATGTTTCAAAGGTGAAG | 9 |
| Sdc1-exon4_5-Forward | GGTGCTTCTCAGAGCCTTT | 10 |
| Sdc1-exon4_5-Reverse | AGGCACACAGCAAAGATGA | 11 |
| Clk3-exon4_5-Forward | GGCGATTGGCTCCAAGA | 12 |
| Clk3-exon4_5-Reverse | AGCACTCCACCACCTTG | 13 |
| Clk3-exon9_10-Forward | CCATGAACATCACACCACCA | 14 |
| Clk3-exon9_10-Reverse | CTCAAAGAGAATGCAGCCGATA | 15 |
| Fam133b-exon6_7-Forward | TCTGATTCTTCCAGCAGTTCTT | 16 |
| Fam133b-exon6_7-Reverse | AGGAGACTTATGGCAACGG | 17 |
| Fam133b-exon7_8-Forward | CGTTGCCATAAGTCTCCTGA | 18 |
| Fam133b-exon7_8-Reverse | TTTCTCTTTCAGTTACATCCTTGG | 19 |
| Gas5-exon4_6-Forward | GTCAGGAAGCTGGATAACAGAG | 20 |
| Gas5-exon4_6-Reverse | AGCCTCAAACTCCACCATTT | 21 |
| Gas5-exon6_8-Forward | GGTGGAGTTTGAGGCTGGATA | 22 |
| Gas5-exon6_8-Reverse | CCAAGCAAGCCAGCCAA | 23 |

### Example 1

### Identification of DDX41 VUS

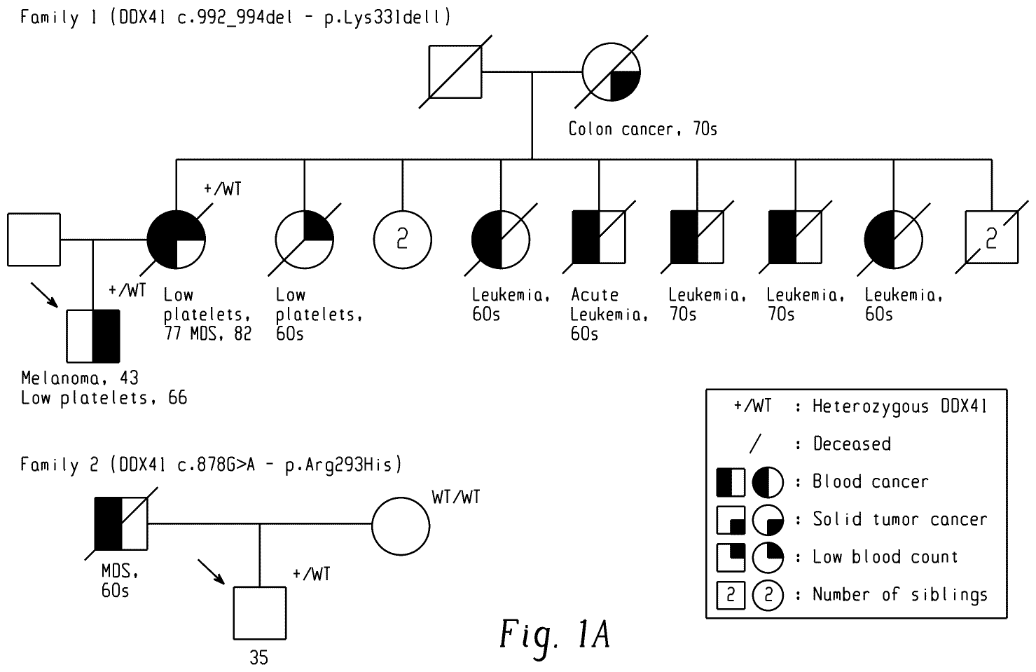

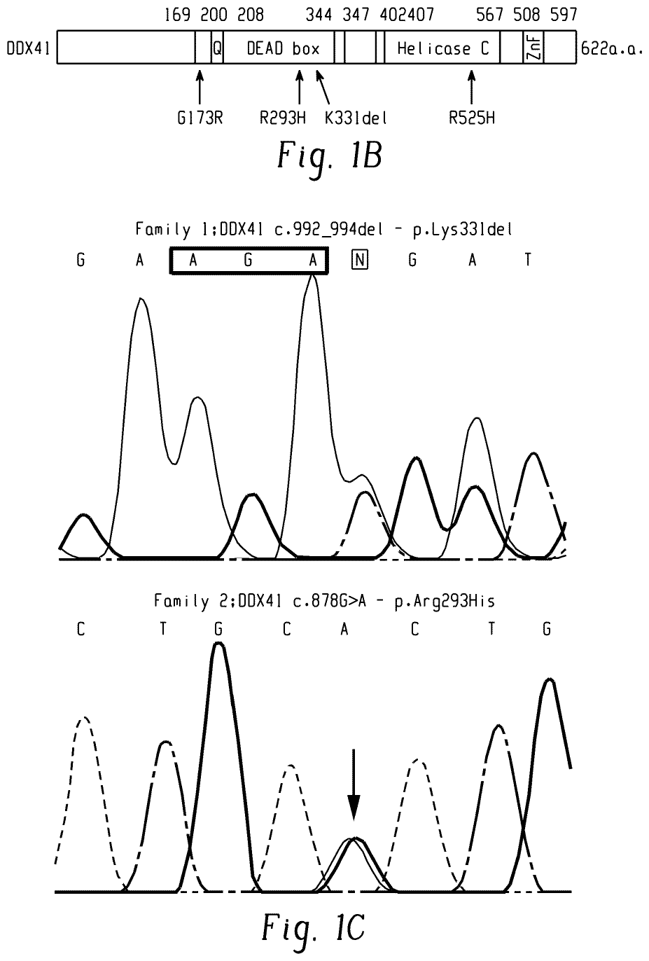

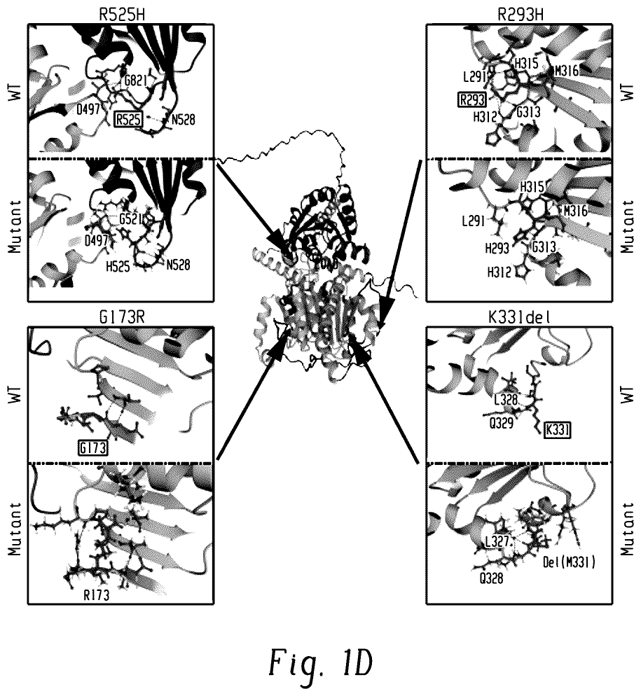

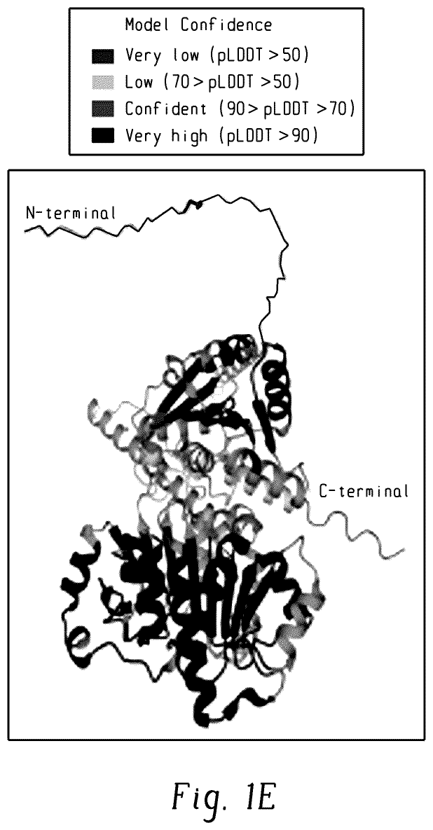

Two rare DDX41 variants of uncertain significance were identified in two unrelated families (FIG. 1A). In family 1 (FIG. 1A upper), the proband with p.Lys33 ldel was diagnosed with melanoma at age 43 and low platelets in the context of a normal bone marrow examination at age 66. His mother was diagnosed with low platelets at age 77 and MDS with excess blasts-1 with a normal karyotype and DDX41 p.Lys331del (VAF 83%) at age 82. Her three brothers and three sisters were diagnosed with low platelets or leukemia after their 60s. In family 2 (FIG. 1A lower), the proband carried germline p.Arg293His. His father was diagnosed with normal karyotype MDS at age 62. He quickly progressed to AML and passed away after allogeneic stem cell transplantation. Both variants occur within the well-conserved DEAD-box domain (FIG. 1B). Sanger sequencing of genomic DNA confirmed DDX41 sequence variation (FIG. 1C). These residues are evolutionarily conserved.

To predict whether deletion or substitution of these residues may disrupt DDX41 three-dimensional structure, AlphaFold was utilized. As with the previously reported DDX41 recurrent germline variant (Glyl73Arg) and the common somatic variant (Arg525His), the gross structural features of the variants are predicted to resemble wild type (WT) DDX41 (FIG. 1D). The modeling predicts that DDX41 Arg293 forms hydrogen bonds surrounding Leu291, His315, Met316, His312, and Gly313 residues. With the histidine substitution of this residue (R293H), the guanidino group is replaced with an imidazole ring, which is predicted to abrogate hydrogen bonding with Leu291, His312, and Gly313 residues (FIG. 1E, upper right). Hydrogen bond disruption was also predicted for the p.Arg525His (R525H) variant. While Arg525 is predicted to form hydrogen bonds with Gly521, Asp497, and Asn528, the Arg525His variant might lose hydrogen bonds with Gly521 and Asp497 (FIG. 1E, upper left). Lys331 can form hydrogen bonds with Leu328 and Gln329. The lysine 331 deletion (K331del) is predicted to disrupt hydrogen bonding with Gln329 (FIG. 1E, lower right). Since intramolecular hydrogen bonds stabilize protein conformations, in principle, altered intramolecular interactions may alter DDX41 function. Although the structural predictive analysis did not reveal whether the variant was pathogenic, based on the prediction of an altered intramolecular interaction, it seems logical that this could affect DDX41 function.

### Example 2

### Production of DDX41+/− Clonal Lines

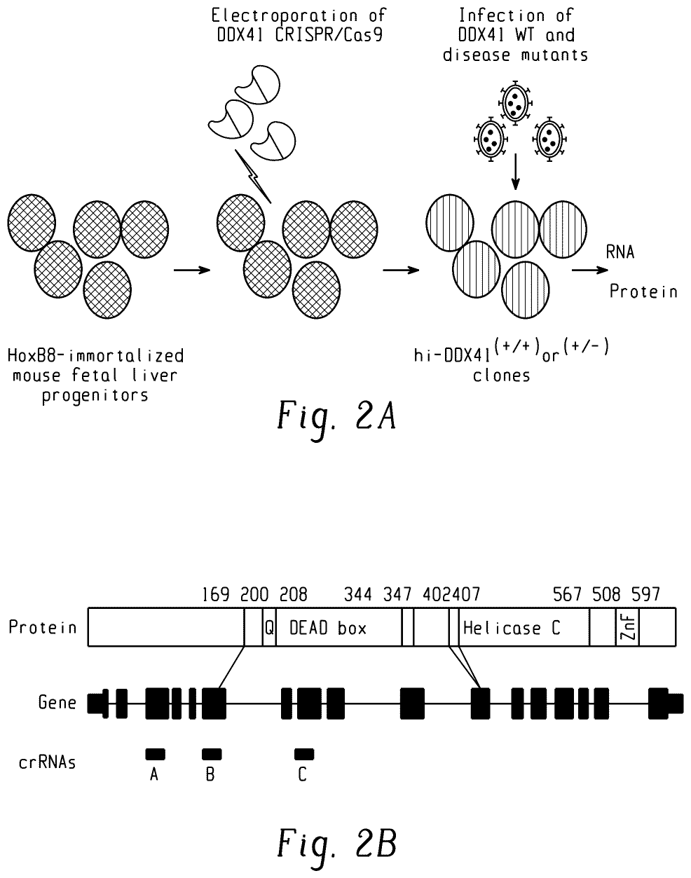

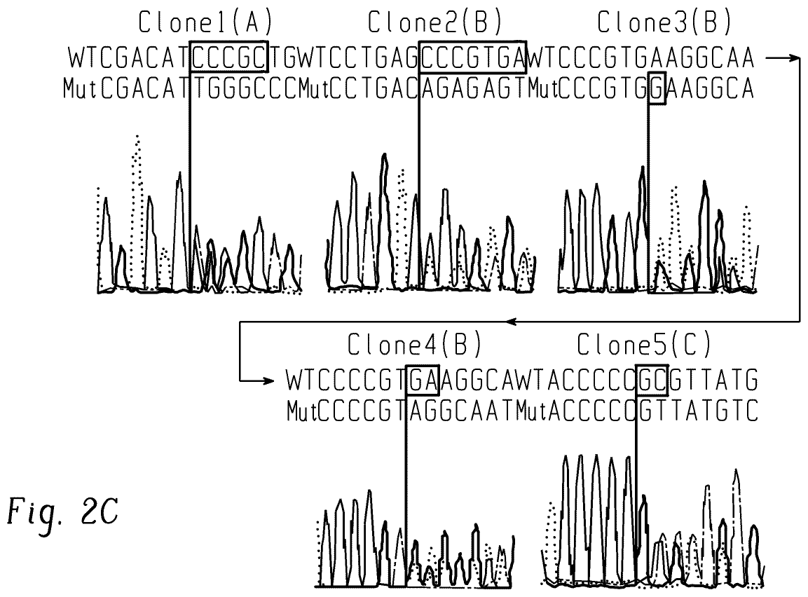

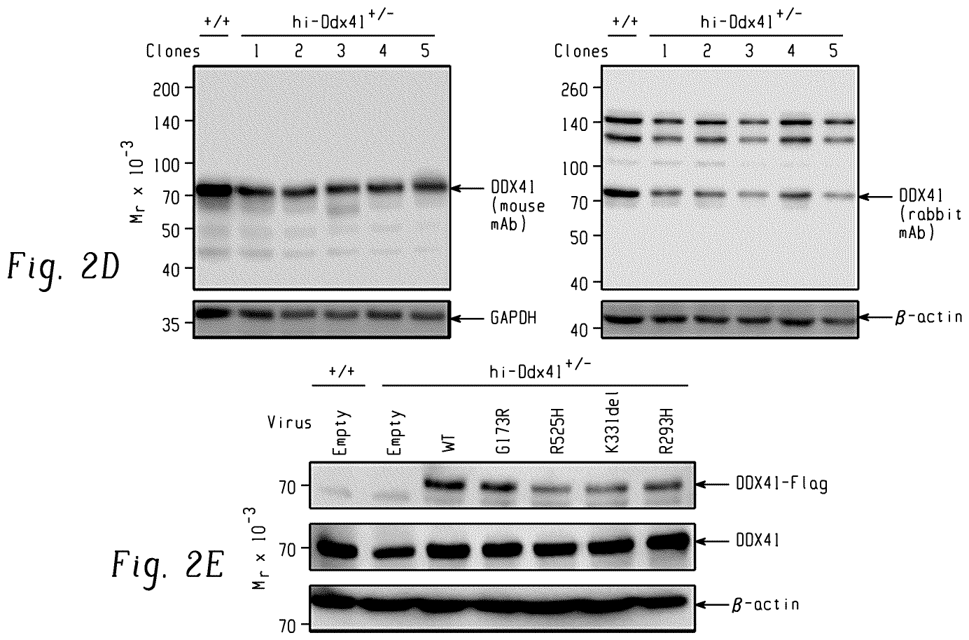

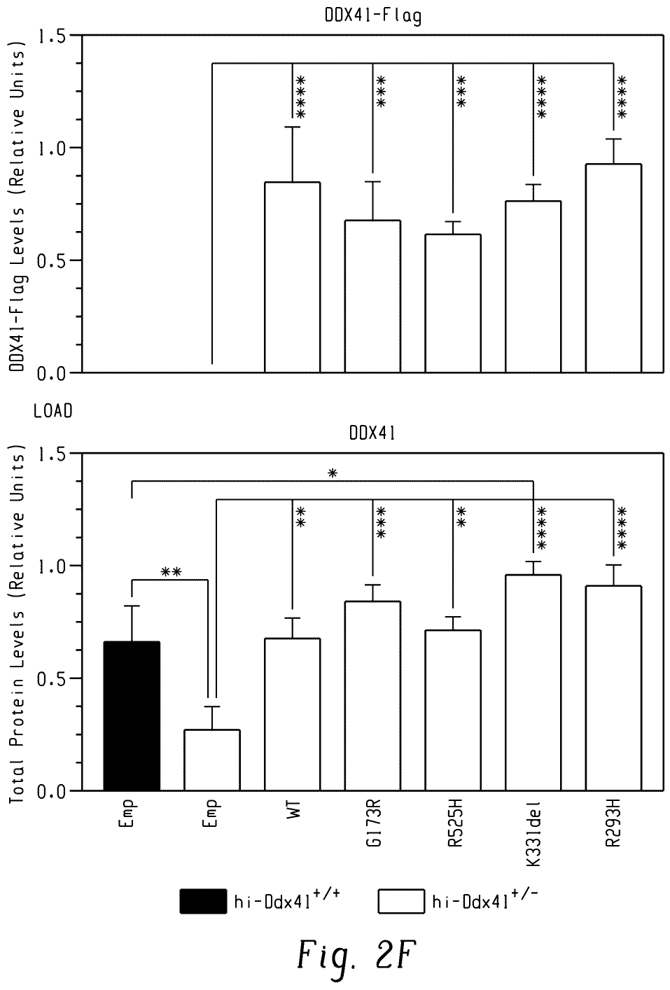

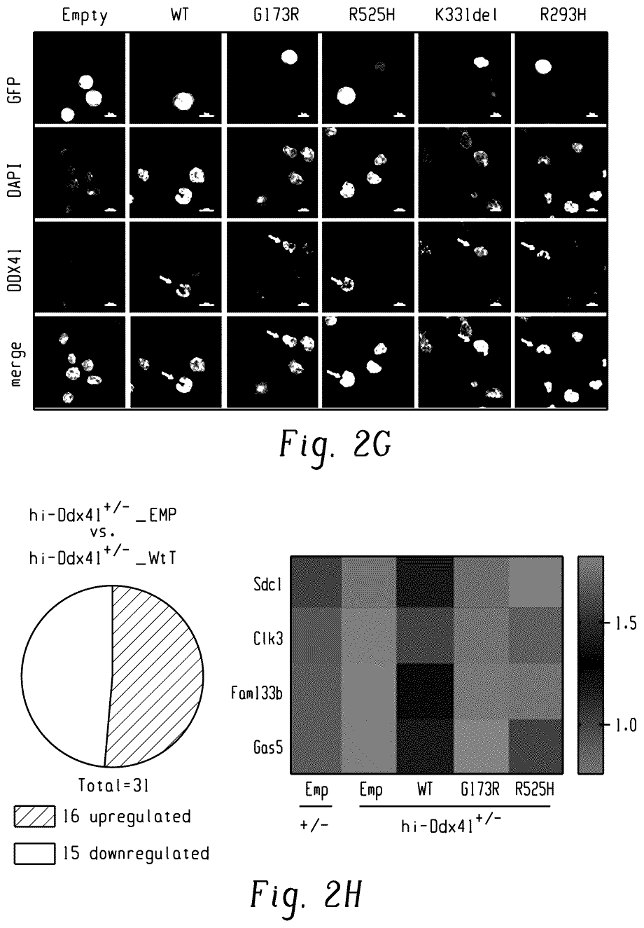

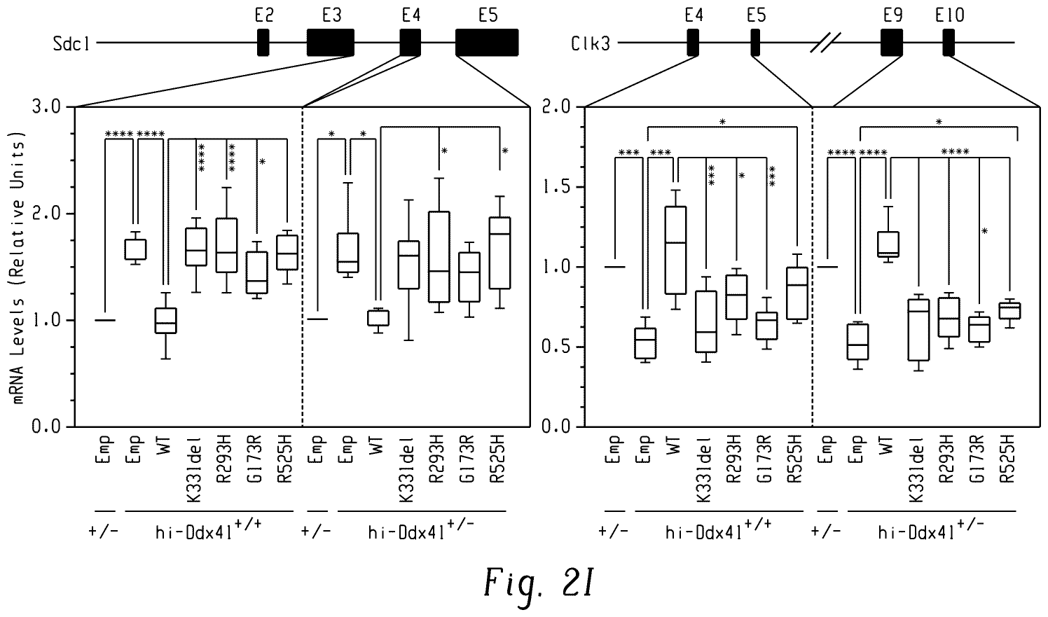

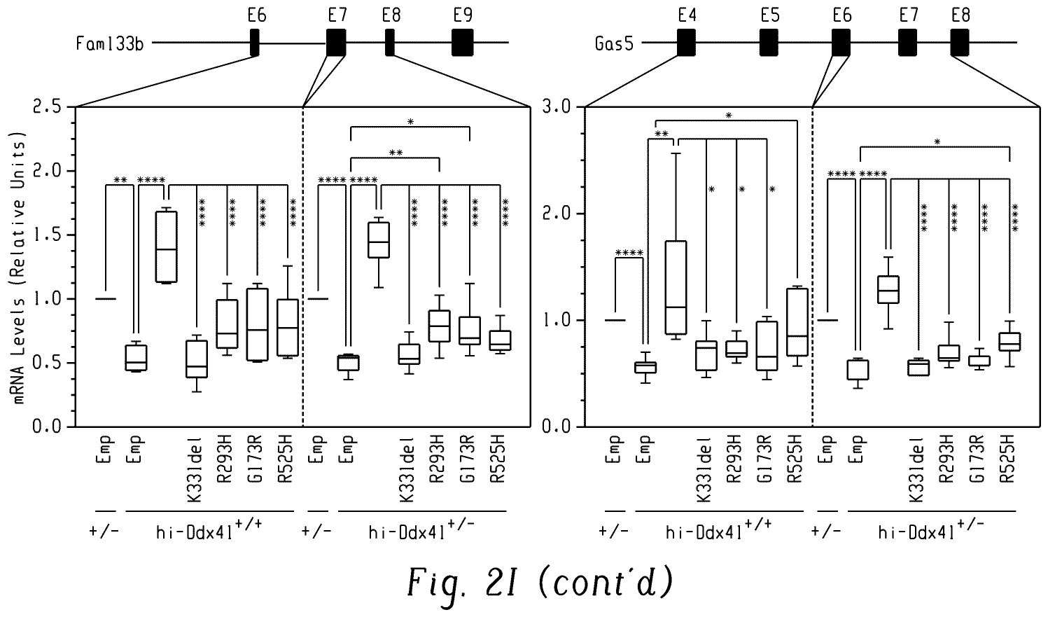

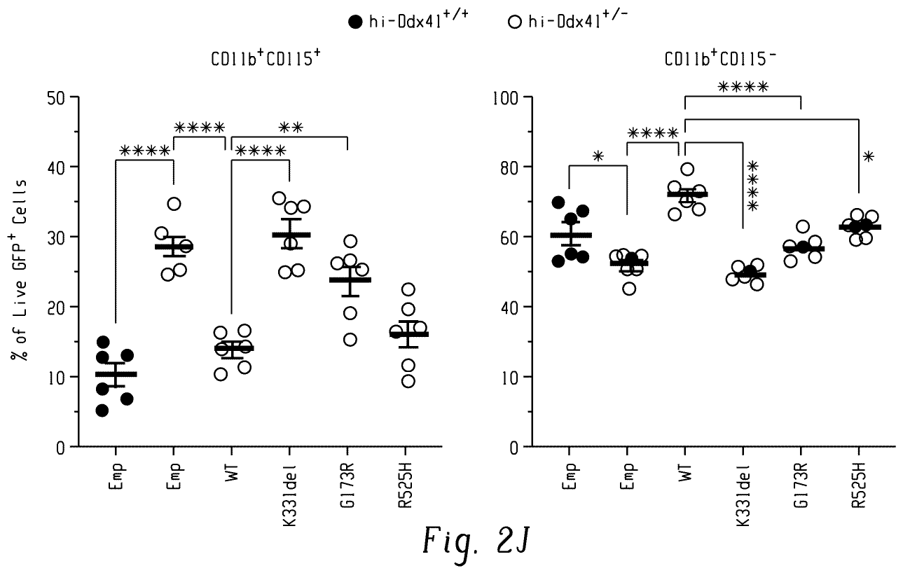

Myeloid malignancy occurring in the context of DDX41 germline variants feature a late age of onset and often acquire an additional somatic DDX41 mutation. Given these attributes and lethality in homozygous knockout mice prior studies to dissect DDX41 mechanisms have been conducted with heterozygous knockout mice, in conjunction with aging or the acquisition of a somatic mutation. As ascribing mechanistic consequences of variants in proteins that profoundly influence cellular functions can be complex, an assay was developed to enable facile mechanistic dissection and clinical curation of DDX41 variants (FIG. 2A) as a complement to complex in vivo strategies. The assay utilizes Hoxb8-immortalized mouse fetal liver progenitor cells, which exhibit a normal myeloid progenitor cell phenotype. These cells were engineered with CRISPR-Cas9 to generate Ddx41^{+/-} clonal lines with reduced endogenous DDX41 protein levels. As Ddx4/-nullizygous mice are lethal, our system, in which DDX41 protein is approximately 50% lower than that of hi-Ddx41^{+/+} cells, minimizes the potential for deleterious effects on cellular functions and provides a model for human DDX41 heterozygous variants. WT or mutant DDX41 proteins can be expressed at near-physiological levels and functional consequences e.g., impact on transcript levels, quantified. The crRNA target sequences resided upstream of the exon encoding functional domains (exon 3 (A)), or within exons encoding the N-terminal DEAD box domain (exon 6 (B) and exon 8 (C), FIG. 2B). The Ddx41+/− cells were validated by Sanger sequencing of genomic DNA at and surrounding the target sequence (FIG. 2C). Semi-quantitative western blotting revealed reduced DDX41 levels in hi-Ddx41^{+/-} relative to hi-Ddx41^{+/+} cells (FIG. 2D). hi-Ddx41^{+/+} and hi-Ddx41^{+/-} cells were infected with retrovirus expressing GFP alone (Empty) or GFP with DDX41 or myeloid malignancy associated DDX41 variants. Because DDX41 protein sequence is highly conserved (99%) between human and mice, human DDX41 was utilized for genetic rescue. After two days, GFP-positive cells were sorted and analyzed by western blotting (FIG. 2E, F). Importantly, the proteins were not overexpressed, as the aggregate level of DDX41 and the DDX41 variant in hi-Ddx41^{+/-} cells resembled that of hi-Ddx41^{+/+} cells (FIG. 2F). While an alternative strategy to analyze DDX41 variants would involve variant knock-in at the endogenous DDX41 locus, this assay allows for multiple variants to be analyzed in the same cellular environment without potential complications from differential genetic influences arising from the use of multiple knock-in lines.

DDX41 has three putative NLS sequences and localizes to the nucleus in the steady-state. All disease-associated variants tested in the genetic-rescue assay were nuclear-localized, resembling DDX41. Thus, the sequence variation did not overtly alter subcellular localization (FIG. 2G). In addition, the cell cycle status of hi-Ddx41^{+/+} and hi-Ddx41^{+/-} cells expressing DDX41 or variants was indistinguishable. To identify DDX41-sensitive molecular processes in hi-Ddx41+/− cells that can be leveraged to quantitatively compare DDX41 and variant activities, RNA-seq was integrated into the rescue system (FIG. 2H). DDX41 expression in hi-Ddx41^{+/-} cells significantly altered the abundance of a restricted ensemble of transcripts; 16 and 15 were upregulated and downregulated, respectively (FIG. 2H, left).

To prioritize candidates from among the DDX41-regulated transcripts, mouse specific genes and predicted genes were excluded, and only genes with TPM>1 were considered. The transcripts were quantified in hi-Ddx41^{+/-} cells, with or without DDX41 expression, to determine if DDX41 rescues transcript levels. Among these transcripts, it was determined whether they were also regulated by DDX41 disease variants, and four were analyzed further (FIG. 2H, right). Syndecan-1 (Sdc1) RNA was elevated 1.6-fold (p=0.0008) in hi-Ddx41^{+/-} versus hi-Ddx41^{+/+} cells, and DDX41, but not the G173R and R525H variants, decreased its expression (68%, p=0.004). Fam133b, Clk3, and Gas5 mRNA levels were lower in hi-Ddx41^{+/-} versus hi-Ddx41^{+/+} cells. DDX41, but not the mutants, increased Fam133b mRNA levels (1.7-fold, p=0.0149). DDX41 increased Clk3 and Gas5 mRNA levels 1.4- and 1.6-fold (p=0.0098 and 0.0564), while the G173R variant was inactive; R525H had reduced activity (1.2-1.4-fold increase, p=0.1123 and 0.2617).

To determine if the novel variants retain competence to alter the levels of DDX41-regulated transcripts, qRT-PCR was conducted with two primer sets that amplify distinct exons of each gene (FIG. 21). Sdc1 expression was 1.7-fold higher (p<0.0001; E3-4, p=0.0194; E4-5) in hi-Ddx41^{+/-} versus hi-Ddx41^{+/+} cells, and DDX41 decreased its expression. Fam133b, Gas5, and Clk3 expression were 40-50% lower (Fam133b; p=0.0075 (E6-7), p<0.0001 (E7-8), Gas5; p<0.0001 (E4-6 and E6-8), and Clk3; p=0.0009 (E4-5), p<0.0001 (E9-10), respectively) in hi-Ddx41^{+/-} versus hi-Ddx41^{+/+} cells, and DDX41 rescued expression. Consistent with the RNA-seq data, G173R was inactive. R525H was not competent to regulate Sdc1 and Fam133b mRNAs, and its activity to regulate Clk3 and Gas5 was attenuated (Clk3, 24% (p=0.1259, E4-5) and 26% (p<0.0001, E9-10), Gas5, 30% (p=0.259, E4-6) and 39% (p<0.0001, E6-8) reduction). K331del variant did not alter the mRNA levels, and R293H variant activity was attenuated relative to DDX41 (Sdc1, 42% (p<0.0001, E3-4) and 37% (p=0.0477, E4-5), Fam133b, 44% (p=0.003, E6-7) and 45% (p=0.0094, E7-8), Clk3, 29% (p=0.0446, E4-5) and 40% (p<0.0001, E9-10), Gas5, 45% (p=0.022, E4-6) and 47% (p<0.0001, E6-8) reduction).

It was further tested whether DDX41 and variants differed with respect to their activity to regulate hematopoietic differentiation in the genetic rescue assay (FIG. 2J). At an early stage of differentiation, although the wild type and heterozygous cells were morphologically indistinguishable (data not shown), changes in cell surface markers were detectable by flow cytometry (FIG. 2J, data not shown). The immortalized progenitor cells have the potential to undergo monocytic and granulocytic differentiation. Ddx41 haploinsufficiency increased monocytic differentiation (CD11b⁺CD115⁺; 2.2-fold increase, p<0.0001). DDX41 expression in hi-Ddx41^{+/-} cells reduced the monocytic population (CD11b+CD115±; 40% relative to control retrovirus-infected hi-Ddx41^{+/-}, p<0.0001), while promoting granulocytic differentiation (CD11b⁺CD115⁻; 1.3-fold increase, p<0.0001). However, the K331del and G173R variants lacked activity. DDX41 and R525H and G610S exhibited comparable activities (CD11b+CD115±; 30% (R525H, p=0.0012) and 50% (G610S, p<0.0001) reduction relative to control retrovirus-infected hi-Ddx41^{+/-}, CD11b⁺CD115⁻; 1.2-fold (R525H, p=0.001) and 1.5-fold (G610S, p<0.0001) increase). Thus, the germline K331del and G173R variants were overtly defective, whereas G610S, a variant of uncertain significance (data not shown) retained activity, and the common somatic variant R525H retained a subset of activities. This genetic rescue-based differentiation assay complements the transcript quantification assay for curating functional attributes of human DDX41 variants.

### Example 3

### Mechanism of Gene Regulation by DDX41

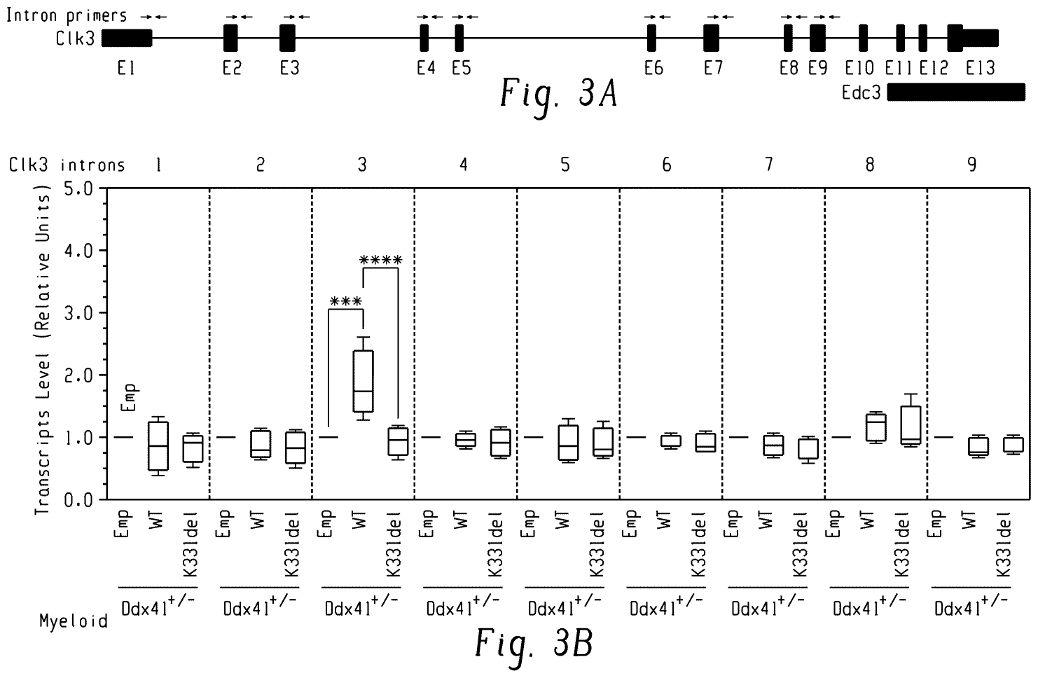

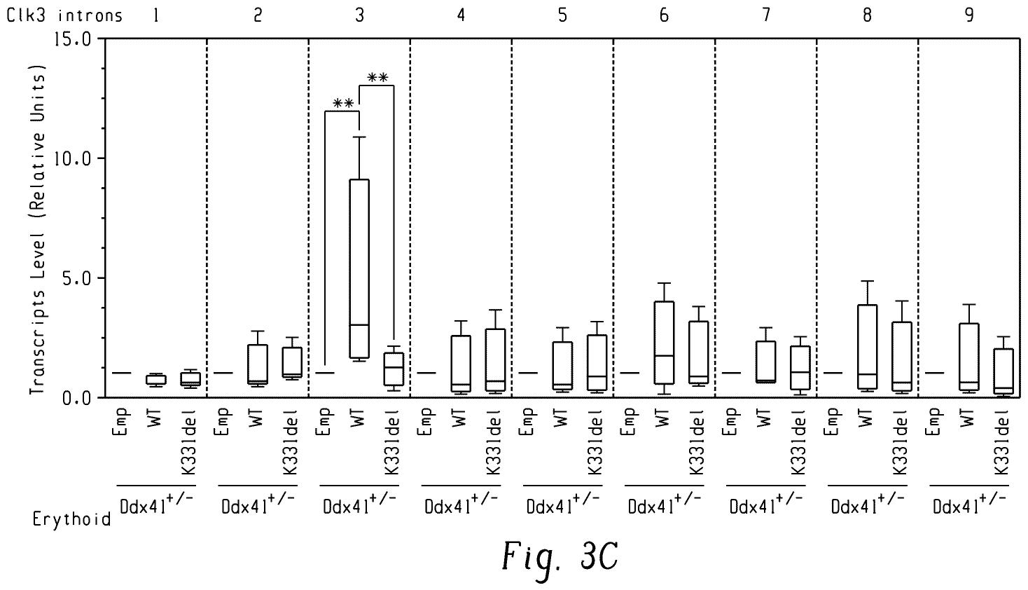

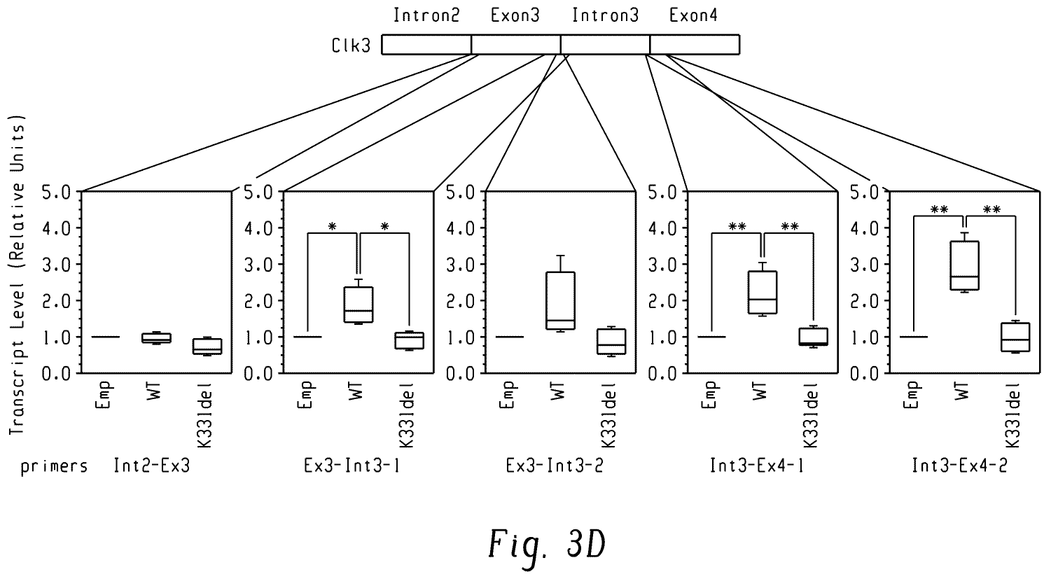

To investigate the mechanism of gene regulation by DDX41, it was first checked whether DDX41 regulates the transcription of Clk3. To measure primary transcript levels of CLK3 by RT-qPCR, primer sets were applied on adjacent exons and introns (FIG. 3A).

### Primer Sets for FIG. 3

SEQUENCE

| | | SEQ |
|---|---|---|
| | | ID |
| | | NO: |
| Clk3-ei1-Forward | TGGGAGACGGTGAGTGT | 24 |
| Clk3-ei1-Reverse | GAAATAGGTCCAAGCCGAGAC | 25 |
| Clk3-ei2-Forward | CATCCCGAAGGGAGCCT | 26 |
| Clk3-ei2-Reverse | AGAGCTCTGGGCCATTCTA | 27 |
| Clk3-ei3-Forward | GTACCAGGTCTTGTAGCAGTG | 28 |
| Clk3-ei3-Reverse | GAGAAAGAGGTGAGGCCTTTAG | 29 |
| Clk3-ei3-2Forward | TCACAAACGCCGTACCAG | 30 |
| Clk3-ei3-2Reverse | GAGAAGCGAGAAAGAGGTGAG | 31 |
| Clk3-ie3-Forward | ACCTCAATCTGTCAATCGGAA | 32 |
| Clk3-ie3-Reverse | CTCCTTGTCATCTTCCACACTC | 33 |
| Clk3-ie3-2Forward | ATTGGTCGGATGGCGTTT | 34 |
| Clk3-ie3-2Reverse | GGCCCTCCTTGTCATCTTC | 35 |
| Clk3-ei4-Forward | TGCCGGATCGGCGATTG | 36 |
| Clk3-ei4-Reverse | GGGTAGAAAGTAGAGAGGTAAAGTT | 37 |
| Clk3-ei5-Forward | GTGGAGTGCTTGGACCAT | 38 |
| Clk3-ei5-Reverse | ACCCAACACATCCAGCAAT | 39 |
| Clk3-ei6-Forward | ATCAAGGAGAAAGACAAGGAAA | 40 |
| Clk3-ei6-Reverse | GCAGCTGGAACTAGAGAATG | 41 |
| Clk3-ei7-Forward | GCTCTGTCATGCCCTTAGAT | 42 |
| Clk3-ei7-Reverse | GGCCTGTAAGACCTTGTAGAAT | 43 |
| Clk3-ei8-Forward | CCAGAGAACATCTTGTTTGTGAAT | 44 |
| Clk3-ei8-Reverse | CTTAGAGCAGGGCCACAC | 45 |
| Clk3-ei9-Forward | ACTACCGCCCACCTGAG | 46 |
| Clk3-ei9-Reverse | TGATAACTGCCAGATGTAAACAATG | 47 |

The exon-intron junction before intron 10, which overlaps with the Edc3 gene, was analyzed by RT-qPCR. Interestingly, only the intron 3 region was increased by 1.7-fold by DDX41 (p<0.001), but not by the pathogenic variant (K331del) in hi-Ddx41^{+/-}(FIG. 3B) In addition, a distinct cellular system (G1E-ER-GATA1 pro-erythroblast, which is relevant to DDX41 actions on the erythroid system in humans) was developed that reiterates the conclusions about the merits of the genetic rescue approach to identify pathogenic variants of DDX41. Consistent with myeloid progenitor (hi-progenitor), DDX41 increased the intron 3 contained transcript by 2.5-fold (p<0.01), whereas K331del did not have an effect (FIG. 3C) Moreover, an intron 3-specific increase was confirmed using two sets of additional primers on the opposite end of intron (3′ of intron 3 and 5′ of exon 4; 2- (primer set 1, p<0.01) and 2.7- (primer set 2, p<0.01) fold (FIG. 3D). These data indicate that DDX41, but not the pathogenic variant (K331del), controls cellular transcript levels by promoting the inclusion of select introns into what should be the fully processed mRNA. The intron-retained transcript is predicted to be stuck in the nucleus. In the case of an inactive variant, it does not do this, and these accumulated transcripts are likely to serve as a reservoir for subsequent use when the cell has a higher demand for protein production.

In conclusion, a genetic rescue assay was developed that quantitatively discriminated activities of DDX41 and myeloid malignancy-associated DDX41 genetic variants. The analyses described herein revealed that the variants were impaired in their intrinsic RNA regulatory activities and to induce monocytic differentiation markers.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. An in vitro genetic rescue assay for identifying functionally defective DDX41 variants, comprising
identifying a DEAD-Box Helicase 41 (DDX41) variant of uncertain significance (VUS),
infecting a first Ddx41^{+/-} cell with a retrovirus expressing the DDX41-VUS,
infecting a second Ddx41^{+/-} cell with a retrovirus expressing a wild type control DDX41,
growing the first and second infected cells in culture for a period of time and quantitating mRNA expression of a DDX41-regulated transcript in both the first and second infected cells after the period of time, wherein the DDX41 related transcript comprises Sdc1, Fam133b, Gas5, Clk3, or a combination thereof,
calculating a differential expression of the DDX41-regulated transcript for the first infected cell compared the second infected cell, and
identifying the DDX-41-VUS as the functionally defective DDX41 variant wherein a change in the differential expression is 1.5-fold or greater,
wherein a Ddx41^{+/-} cell has a DDX41 expression level that is reduced by at least 50% compared to that of a Ddx41^{+/+} cell.

2. The method of claim 1, wherein identifying the DDX-41 VUS comprises identifying the DDX-41 VUS in a patient presenting with cytopenia, bone marrow hypocellularity, erythroid dysplasia, and/or myeloid malignancy, but not diagnosed with acute myeloid leukemia; or identifying the DDX-41 VUS in a database.

3. The method of claim 1, wherein quantitating mRNA expression of a DDX41-regulated transcript comprises reverse-transcriptase PCR.

4. The method of claim 1, further comprising
infecting a third Ddx41^{+/-} cell with a retrovirus expressing a functionally defective variant control DDX41,
growing the third infected cells in culture for a period of time and quantitating mRNA expression of DDX41-regulated genes in the third infected cells after the period of time, wherein the DDX41-regulated genes comprises Sdc1, Fam133b, Gas5, Clk3, or a combination thereof, and
calculating a differential expression of the DDX41-regulated genes for the first infected cell compared to the third infected cell to determine a functionally defective control.

5. The method of claim 1, further comprising
performing flow cytometry on the first infected cells and second infected cells and quantitating a monocytic marker of differentiation of hematopoietic progenitor cells and a granulocytic marker of differentiation of hematopoietic progenitor cells with marker-specific antibodies, wherein a 50% or greater increase or decrease in the quantitative level of the monocytic marker, the granulocytic maker, or both, in the first infected cells compared to the second infected cells identifies the functionally defective DDX41 variant as a pathogenic DDX41 variant.

6. The method of claim 5, wherein the monocytic marker is CD11b⁺CD115⁺ and the granulocytic marker is CD11b⁺CD115⁻.

7. The method of claim 1, wherein Ddx41^{+/-} cells were prepared from Hoxb8-immortalized mouse fetal liver progenitor cells.

8. A method of monitoring a patient for the development/progression of a myeloid malignancy, comprising
determining the presence or absence of a functionally defective DDX41 variant determined according to the method of claim 1 in a sample from the patient,
optionally determining the presence or absence of the functionally defective DDX41 variant in one or more family members of the patient, and
monitoring the patient and optionally the one or more family members for the development of one or more symptoms of myeloid malignancy when the patient and the one or more family members carry the functionally defective DDX41 variant.

9. The method of claim 8, wherein the sample is blood, bone marrow, fibroblasts, or fractional bone marrow hematopoietic stem/progenitor cells.

10. The method of claim 8, wherein the myeloid malignancy is myelodysplastic syndromes (MDS) or acute myeloid leukemia (AML).

11. The method of claim 8, wherein the one or more symptoms of myeloid malignancy comprises fever, fatigue, irregular heartbeat, dizziness, bone pain, frequent nosebleeds, bleeding and swollen gums, bruising on skin, loss of appetite, excessive sweating, shortness of breath, unexplained weight loss, headaches, diarrhea, menorrhagia, slurred speech, confusion, abdominal swelling, pale skin, seizures, vomiting, loss of balance, facial numbness, blurred vision, or a combination thereof.

12. The method of claim 8, wherein when the patient carries the functionally defective DDX41 variant, administering a treatment for the myeloid malignancy.

13. The method of claim 12, wherein the treatment is chemotherapy or a stem cell transplant.

14. The method of claim 8, wherein the subject has a risk factor for myeloid malignancy, the risk factor comprising being a smoker, being over 65 years of age, exposure to chemotherapy or radiation such as radiation therapy, having a history of a blood disorder, a family history of myeloid malignancy, or a combination thereof.

15. The method of claim 8, wherein the functionally defective DDX41 variant is Lys331del.

16. A method of identifying a patient as at risk for the development/progression of a myeloid malignancy, comprising
identifying a functionally defective DDX41 variant in the germline of the patient, wherein the functionally defective DDX41 variant is Lys331del.

17. The method of claim 16, further comprising administering a treatment for the myeloid malignancy.

18. The method of claim 17, wherein the treatment is chemotherapy or a stem cell transplant.


SEQUENCE LISTING (47 sequences)

SEQ ID NO: 1 - RNA, 20 nt, synthetic construct
  source 1..20: mol_type = other RNA; note = crRNA
agatgaggacgacatcccgc

SEQ ID NO: 2 - RNA, 20 nt, synthetic construct
  source 1..20: mol_type = other RNA; note = crRNA
tgatcggcattgccttcacg

SEQ ID NO: 3 - RNA, 20 nt, synthetic construct
  source 1..20: mol_type = other RNA; note = crRNA
ccgcgttatgtcctgagcat

SEQ ID NO: 4 - DNA, 20 bp, synthetic construct
  source 1..20: mol_type = other DNA; note = Primer DDX41 Exon9-Forward
atcatctgcccctcggtaag

SEQ ID NO: 5 - DNA, 20 bp, synthetic construct
  source 1..20: mol_type = other DNA; note = Primer DDX41 Exon9-Reverse
ccccaactaacctccccatt

SEQ ID NO: 6 - DNA, 20 bp, synthetic construct
  source 1..20: mol_type = other DNA; note = Primer DDX41 Exon10-Forward
atacatagggcaggtggtgg

SEQ ID NO: 7 - DNA, 20 bp, synthetic construct
  source 1..20: mol_type = other DNA; note = Primer DDX41 Exon10-Reverse
agctgagcaactgagacaca

SEQ ID NO: 8 - DNA, 20 bp, synthetic construct
  source 1..20: mol_type = other DNA; note = Primer Sdc1-exon3_4-Forward
gaggatggaactgccaatca

SEQ ID NO: 9 - DNA, 22 bp, synthetic construct
  source 1..22: mol_type = other DNA; note = Primer Sdc1-exon3_4-Reverse
cccagatgtttcaaaggtgaag

SEQ ID NO: 10 - DNA, 19 bp, synthetic construct
  source 1..19: mol_type = other DNA; note = Primer Sdc1-exon4_5-Forward
ggtgcttctcagagccttt

SEQ ID NO: 11 - DNA, 19 bp, synthetic construct
  source 1..19: mol_type = other DNA; note = Primer Sdc1-exon4_5-Reverse
aggcacacagcaaagatga

SEQ ID NO: 12 - DNA, 17 bp, synthetic construct
  source 1..17: mol_type = other DNA; note = Primer Clk3-exon4_5-Forward
ggcgattggctccaaga

SEQ ID NO: 13 - DNA, 17 bp, synthetic construct
  source 1..17: mol_type = other DNA; note = Primer Clk3-exon4_5-Reverse
agcactccaccaccttg

SEQ ID NO: 14 - DNA, 20 bp, synthetic construct
  source 1..20: mol_type = other DNA; note = Primer Clk3-exon9_10-Forward
ccatgaacatcacaccacca

SEQ ID NO: 15 - DNA, 22 bp, synthetic construct
  source 1..22: mol_type = other DNA; note = Primer Clk3-exon9_10-Reverse
ctcaaagagaatgcagccgata

SEQ ID NO: 16 - DNA, 22 bp, synthetic construct
  source 1..22: mol_type = other DNA; note = Primer Fam133b-exon6_7-Forward
tctgattcttccagcagttctt

SEQ ID NO: 17 - DNA, 19 bp, synthetic construct
  source 1..19: mol_type = other DNA; note = Primer Fam133b-exon6_7-Reverse
aggagacttatggcaacgg

SEQ ID NO: 18 - DNA, 20 bp, synthetic construct
  source 1..20: mol_type = other DNA; note = Primer Fam133b-exon7_8-Forward
cgttgccataagtctcctga

SEQ ID NO: 19 - DNA, 24 bp, synthetic construct
  source 1..24: mol_type = other DNA; note = Primer Fam133b-exon7_8-Reverse
tttctctttcagttacatccttgg

SEQ ID NO: 20 - DNA, 22 bp, synthetic construct
  source 1..22: mol_type = other DNA; note = Primer Gas5-exon4_6-Forward
gtcaggaagctggataacagag

SEQ ID NO: 21 - DNA, 20 bp, synthetic construct
  source 1..20: mol_type = other DNA; note = Primer Gas5-exon4_6-Reverse
agcctcaaactccaccattt

SEQ ID NO: 22 - DNA, 21 bp, synthetic construct
  source 1..21: mol_type = other DNA; note = Primer Gas5-exon6_8-Forward
ggtggagtttgaggctggata

SEQ ID NO: 23 - DNA, 17 bp, synthetic construct
  source 1..17: mol_type = other DNA; note = Primer Gas5-exon6_8-Reverse
ccaagcaagccagccaa

SEQ ID NO: 24 - DNA, 17 bp, synthetic construct
  source 1..17: mol_type = other DNA; note = Primer Clk3-ei1-Forward
tgggagacggtgagtgt

SEQ ID NO: 25 - DNA, 21 bp, synthetic construct
  source 1..21: mol_type = other DNA; note = Primer Clk3-ei1-Reverse
gaaataggtccaagccgagac

SEQ ID NO: 26 - DNA, 17 bp, synthetic construct
  source 1..17: mol_type = other DNA; note = Primer Clk3-ei2-Forward
catcccgaagggagcct

SEQ ID NO: 27 - DNA, 19 bp, synthetic construct
  source 1..19: mol_type = other DNA; note = Primer Clk3-ei2-Reverse
agagctctgggccattcta

SEQ ID NO: 28 - DNA, 21 bp, synthetic construct
  source 1..21: mol_type = other DNA; note = Primer Clk3-ei3-Forward
gtaccaggtcttgtagcagtg

SEQ ID NO: 29 - DNA, 22 bp, synthetic construct
  source 1..22: mol_type = other DNA; note = Primer Clk3-ei3-Reverse
gagaaagaggtgaggcctttag

SEQ ID NO: 30 - DNA, 18 bp, synthetic construct
  source 1..18: mol_type = other DNA; note = Primer Clk3-ei3-2Forward
tcacaaacgccgtaccag

SEQ ID NO: 31 - DNA, 21 bp, synthetic construct
  source 1..21: mol_type = other DNA; note = Primer Clk3-ei3-2Reverse
gagaagcgagaaagaggtgag

SEQ ID NO: 32 - DNA, 21 bp, synthetic construct
  source 1..21: mol_type = other DNA; note = Primer Clk3-ie3-Forward
acctcaatctgtcaatcggaa

SEQ ID NO: 33 - DNA, 22 bp, synthetic construct
  source 1..22: mol_type = other DNA; note = Primer Clk3-ie3-Reverse
ctccttgtcatcttccacactc

SEQ ID NO: 34 - DNA, 18 bp, synthetic construct
  source 1..18: mol_type = other DNA; note = Primer Clk3-ie3-2Forward
attggtcggatggcgttt

SEQ ID NO: 35 - DNA, 19 bp, synthetic construct
  source 1..19: mol_type = other DNA; note = Primer Clk3-ie3-2Reverse
ggccctccttgtcatcttc

SEQ ID NO: 36 - DNA, 17 bp, synthetic construct
  source 1..17: mol_type = other DNA; note = Primer Clk3-ei4-Forward
tgccggatcggcgattg

SEQ ID NO: 37 - DNA, 25 bp, synthetic construct
  source 1..25: mol_type = other DNA; note = Primer Clk3-ei4-Reverse
gggtagaaagtagagaggtaaagtt

SEQ ID NO: 38 - DNA, 18 bp, synthetic construct
  source 1..18: mol_type = other DNA; note = Primer Clk3-ei5-Forward
gtggagtgcttggaccat

SEQ ID NO: 39 - DNA, 19 bp, synthetic construct
  source 1..19: mol_type = other DNA; note = Primer Clk3-ei5-Reverse
acccaacacatccagcaat

SEQ ID NO: 40 - DNA, 22 bp, synthetic construct
  source 1..22: mol_type = other DNA; note = Primer Clk3-ei6-Forward
atcaaggagaaagacaaggaaa

SEQ ID NO: 41 - DNA, 20 bp, synthetic construct
  source 1..20: mol_type = other DNA; note = Primer Clk3-ei6-Reverse
gcagctggaactagagaatg

SEQ ID NO: 42 - DNA, 20 bp, synthetic construct
  source 1..20: mol_type = other DNA; note = Primer Clk3-ei7-Forward
gctctgtcatgcccttagat

SEQ ID NO: 43 - DNA, 22 bp, synthetic construct
  source 1..22: mol_type = other DNA; note = Primer Clk3-ei7-Reverse
ggcctgtaagaccttgtagaat

SEQ ID NO: 44 - DNA, 24 bp, synthetic construct
  source 1..24: mol_type = other DNA; note = Primer Clk3-ei8-Forward
ccagagaacatcttgtttgtgaat

SEQ ID NO: 45 - DNA, 18 bp, synthetic construct
  source 1..18: mol_type = other DNA; note = Primer Clk3-ei8-Reverse
cttagagcagggccacac

SEQ ID NO: 46 - DNA, 17 bp, synthetic construct
  source 1..17: mol_type = other DNA; note = Primer Clk3-ei9-Forward
actaccgcccacctgag

SEQ ID NO: 47 - DNA, 25 bp, synthetic construct
  source 1..25: mol_type = other DNA; note = Primer Clk3-ei9-Reverse
tgataactgccagatgtaaacaatg